(19) United States
(12) Patent Application Publication
(10) Pub. No.: US 2018/0375053 A1
(43) Pub. Date: Dec. 27, 2018

(54) PHOSPHORESCENT ORGANIC ELECTROLUMINESCENCE DEVICES

(71) Applicants: KUNSHAN GO-VISIONOX OPTO-ELECTRONICS CO., LTD., KunShan City, Jiangsu (KR); TSINGHUA UNIVERSITY, Beijing (CN)

(72) Inventors: Lian DUAN, Beijing (CN); Dongdong ZHANG, Beijing (CN); Song LIU, Beijing (CN); Fei ZHAO, Beijing (CN)

(73) Assignees: KUNSHAN GO-VISIONOX OPTO-ELECTRONICS CO., LTD., KunShan City, Jiangsu (KR); TSINGHUA UNIVERSITY, Beijing (CN)

(21) Appl. No.: 15/781,439

(22) PCT Filed: Nov. 25, 2016
(86) PCT No.: PCT/CN2016/107232

§ 371 Date: Jun. 4, 2018

(30) Foreign Application Priority Data
Dec. 18, 2015 (CN) 201510952274.5

(51) Int. Cl. H01L 51/50; H01L 51/00

(52) U.S. Cl. CPC H01L 51/5096; H01L 51/005; H01L 51/5052; H01L 51/0085; H01L 51/5016; H01L 51/0081

(57) ABSTRACT

The present invention discloses a phosphorescent organic electroluminescence device including a hole transport layer, a luminescent layer and an electron transport layer, which are successively laminated. The luminescent layer has a double-layer structure comprising a hole transport material layer and an electron transport material layer. The hole transport material layer is arranged between the hole transport layer and the electron transport material layer. The electron transport material layer is arranged between the hole transport material layer and the electron transport layer. An exciplex is formed on the interface of contact between the hole transport material layer and the electron transport material layer. The hole transport material layer includes a host material, the host material being a material having hole transport capability; and the electron transport material layer includes a host material and a phosphorescent dye doped in the host material, the host material being a material having electron transport capability.

### TECHNICAL FIELD

The present invention belongs to the field of organic electroluminescence devices, and specifically relates to a phosphorescent organic electroluminescence device.

### BACKGROUND ART

Organic electroluminescence devices have attracted extensive attention due to their features of thinness, large area, full curing and flexibility, and their potential in solid-state lighting sources and liquid crystal backlights has become a focus of research.

As early as in 1950s, Bernanose. A et al. began the research on organic electroluminescence devices (OLEDs). Anthracene single crystal wafer was adopted as the original research materials. Because of large thickness of the single crystal wafer, the driving voltage required is very high. Until 1987, C. W. Tang and Vanslyke from Eastman Kodak Company proposed an organic small molecule electroluminescence device having the structure ITO/Diamine/Alq₃/Mg:Ag. It was reported that when this device worked at a working voltage of 10 V, the luminance reached 1000 cd/m2 and the external quantum efficiency reached 1.0%. The research on electroluminescence has drawn wide attention from scientists, and it is possible for OLEDs to be used in display. Since then, the research on OLEDs and industrialization of OLEDs has been started. An organic luminescent material system includes a fluorescent system and a phosphorescent luminescent system, in which the fluorescent system utilizes singlet state exciton energy alone, and the phosphorescent system can utilize both singlet and triplet state exciton energy.

In a phosphorescent device in which a conventional host exists, energy is transferred from the host at triplet state to a phosphorescent guest at triplet state by short-range Dexter energy transfer, resulting in a higher doping concentration (10 wt %-30 wt %) of the phosphorescent material; although the high doping concentration may reduce the distance between the host and guest and promote complete transfer of energy, the excessive high doping concentration may cause the decrease of device efficiency, also, as the phosphorescent material is made of a noble metal, the high phosphorescent dye doping concentration may increase the cost.

### Technical Problems

The technical problem to be solved by the present invention is that: in a phosphorescent device in which a conventional host exists, energy is transferred from the host at triplet state to a phosphorescent guest at triplet state by short-range Dexter energy transfer, resulting in a higher doping concentration (10 wt %-30 wt %) of the phosphorescent material; the excessive high doping concentration may cause the decrease of device efficiency; also, as the phosphorescent material is made of a noble metal, the high phosphorescent dye doping concentration may increase the cost.

### Technical Solutions

In order to solve the above technical problem, the present invention provides a novel phosphorescent organic electroluminescence device. The phosphorescent organic electroluminescence device includes a hole transport material layer and an electron transport material layer, an exciplex is formed on the interface of the two layers, so that energy is transferred from triplet state of the host material to singlet state of the host by reverse intersystem crossing and then to triplet state of the phosphorescent guest by long-range Föster energy transfer, thereby reducing the doping concentration of a phosphorescent dye.

The phosphorescent organic electroluminescence device provided by the present invention includes a hole transport layer, a luminescent layer and an electron transport layer, which are successively laminated; the luminescent layer has a double-layer structure comprising a hole transport material layer and an electron transport material layer; the hole transport material layer is arranged between the hole transport layer and the electron transport material layer; the electron transport material layer is arranged between the hole transport material layer and the electron transport layer; and an exciplex is formed on the interface of contact between the hole transport material layer and the electron transport material layer;

the hole transport material layer includes a host material, the host material being a material having hole transport capability;

the electron transport material layer includes a host material and a phosphorescent material doped in the host material, the host material being a material having electron transport capability;

wherein, the first triplet state energy level of the material having hole transport capability is higher than the first singlet state energy level of the exciplex, with an energy gap between the material having electron transport capability and the exciplex being more than or equal to 0.2 eV; and the absolute value of HOMO energy level of the material having hole transport capability is less than or equal to 5.3 eV;

the first triplet state energy level of the material having electron transport capability is higher than the first singlet state energy level of the exciplex, with an energy gap between the material having electron transport capability and the exciplex being more than 0.2 eV; and the absolute value of LUMO energy level of the material having electron transport capability is more than 2.0 eV; the difference in LUMO energy level between the material having hole transport capability and the material having electron transport capability is more than 0.3 eV, and the difference in HOMO energy level between the material having hole transport capability and the material having electron transport capability is more than 0.2 eV; and

the first singlet state energy level of the exciplex is higher than the first triplet state energy level of the phosphorescent material.

Preferably, the difference in LUMO energy level between the material having hole transport capability and the material having electron transport capability is more than or equal to 0.4 eV.

Wherein, the phosphorescent dye in the luminescent layer account for 1 wt % to 10 wt %, preferably 3 wt %.

Preferably, the thickness ratio of the hole transport material layer and the electron transport material layer is 1:1 to 1:5, preferably 1:3.

Preferably, the material having electron transport capability is one or more of compounds of the formulae:

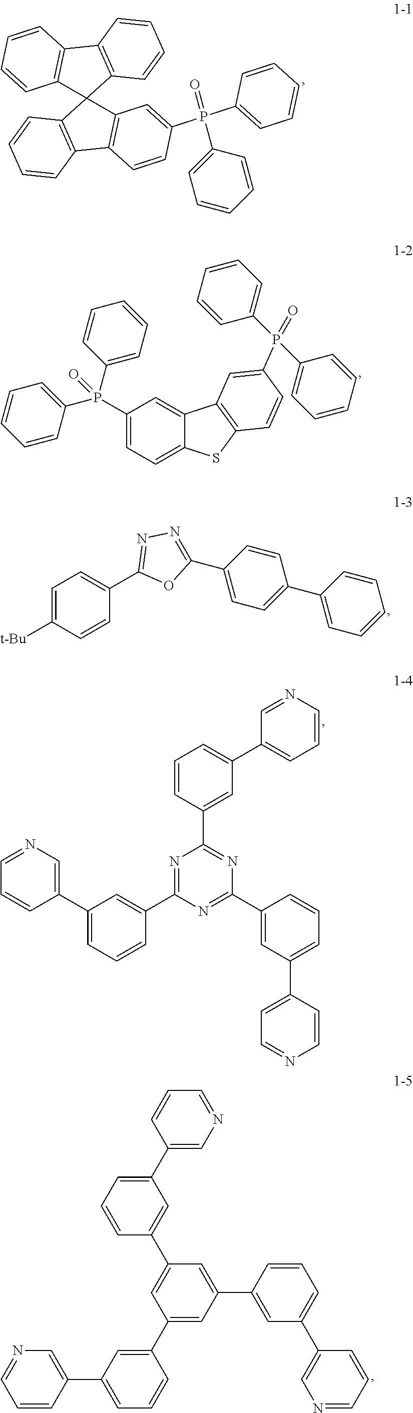 [SMILES: O=P(c1ccccc1)(c1ccccc1)c1ccc2c(c1)C1(c3ccccc3-c3ccccc31)c1ccccc1-2 ; O=P(c1ccccc1)(c1ccccc1)c1ccc2sc3ccc(P(=O)(c4ccccc4)c4ccccc4)cc3c2c1 ; CC(C)(C)c1ccc(-c2nnc(-c3ccc(-c4ccccc4)cc3)o2)cc1 ; c1cncc(-c2cccc(-c3nc(-c4cccc(-c5ccncc5)c4)nc(-c4cccc(-c5cccnc5)c4)n3)c2)c1 ; c1cncc(-c2cccc(-c3cc(-c4cccc(-c5cccnc5)c4)cc(-c4cccc(-c5cccnc5)c4)c3)c2)c1]

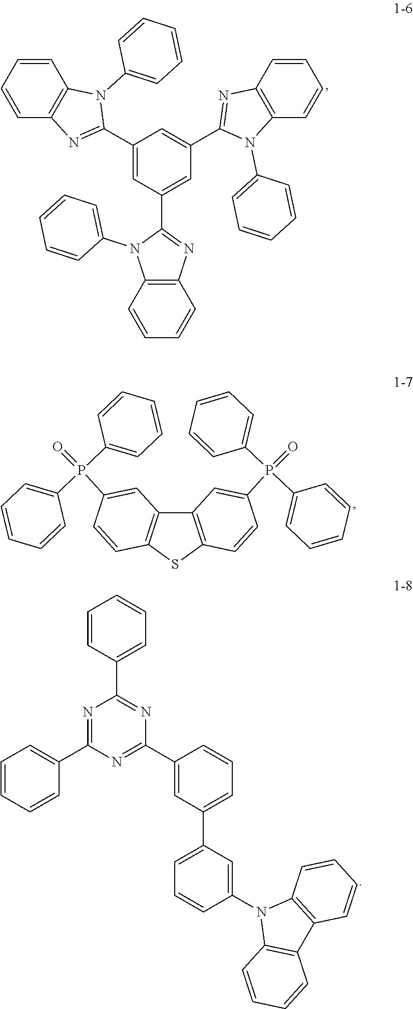

Preferably, the material having hole transport capability is one or more of compounds of the following formulae:

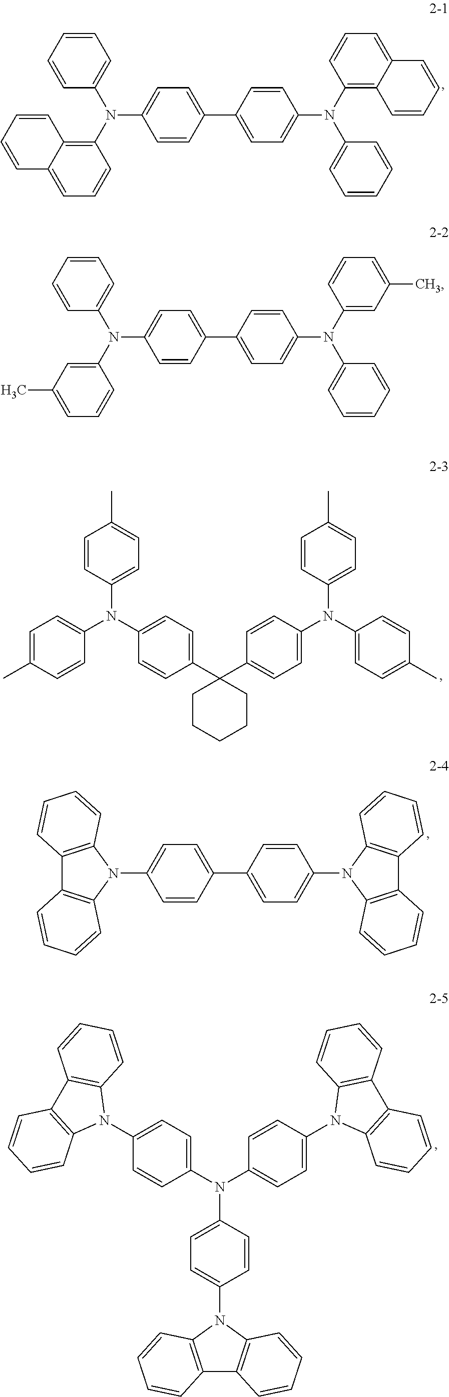

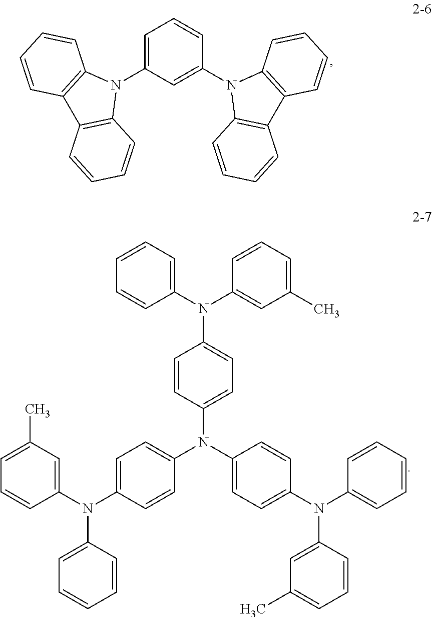

The phosphorescent organic electroluminescence device of the present invention includes an anode, a hole transport layer, the hole transport material layer, the electron transport material layer, an electron transport layer and a cathode, which are successively arranged on a substrate. The anode and the hole transport layer has a hole injection layer arranged therebetween.

### Advantageous Effects

The present invention has the following advantages:

In the phosphorescent organic electroluminescence device of the present invention, the luminescent layer is a double-layer structure and the exciplex is formed on the interface of the hole transport material layer and the electron transport material layer, the exciplex is a TADF exciplex having a thermally activated delayed fluorescence effect, and the triplet state energy of the TADF exciplex is transferred to the singlet state energy by reverse intersystem crossing and then transferred to the triplet state energy of the dopant dye; so that the triplet state energy of the host material and dopant dye in the device can be fully utilized, thereby increasing the device efficiency; and the energy transfer process and luminescent process of thermally activated delayed fluorescence occur in different materials (named as thermally activated sensitization process), so that the problem of significant roll-off under high luminance conditions can be effectively solved, thereby further improving the stability of the device.

In the phosphorescent organic electroluminescence device of the present invention, the luminescent layer utilizes the exciplex to reduce the doping concentration of the phosphorescent dye and also maintain long lifetime and high efficiency.

### BRIEF DESCRIPTION OF THE DRAWINGS

FIG. 1 schematically shows a structure of an organic electroluminescence device in accordance to the invention.

FIG. 2 schematically shows energy transfer of a luminescent layer of an organic electroluminescence device in accordance to the invention.

### PREFERRED EMBODIMENTS OF THE INVENTION

The present invention will be further described below with reference to the accompanying drawings and specific embodiments, so that those skilled in the art can better understand and implement the present invention, however, the embodiments are not intended to limit the present invention.

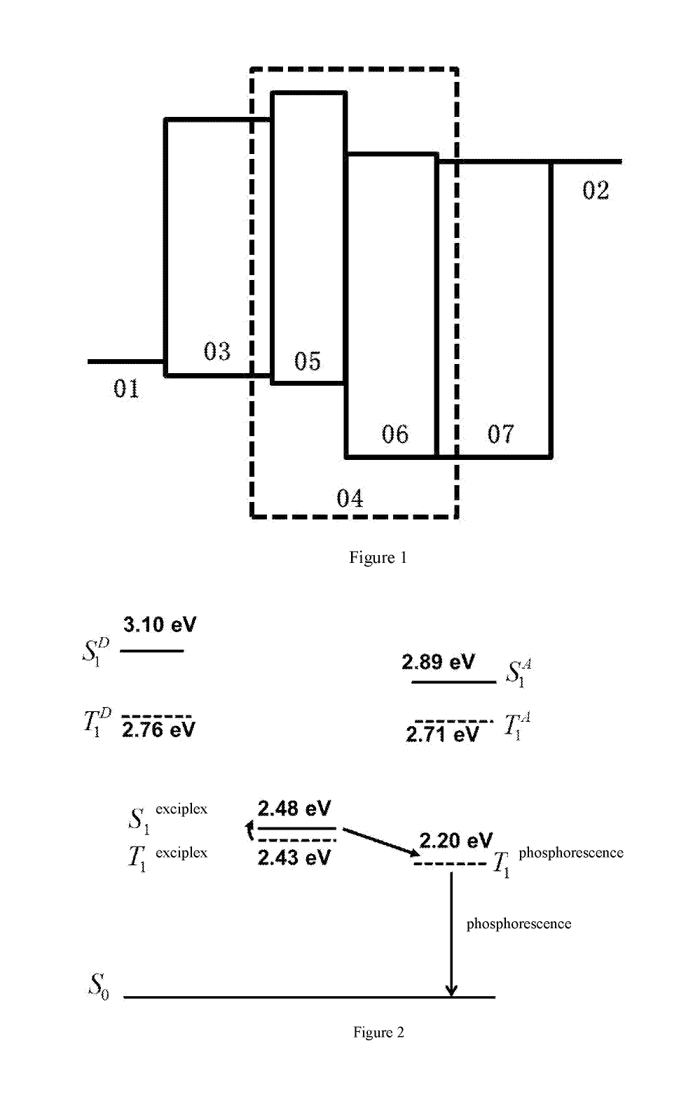

As shown in FIG. 1, the organic electroluminescence device of the invention includes successively depositing on the substrate an anode 01, a hole transport layer 03, a luminescent layer 04, an electron transport layer 07 and a cathode 02 in a way of laminating one by one, wherein the luminescent layer includes a hole transport material layer 05 and an electron transport material layer 06.

The luminescent layer is a double-layer structure formed by the hole transport material layer 05 and the electron transport material layer 06; the hole transport material layer 05 is arranged between the hole transport layer and the electron transport material layer; the electron transport material layer 06 is arranged between a donor layer and the hole transport layer 07; and an exciplex is formed on the interface of contact between the hole transport material layer 05 and the electron transport material layer 06;

the hole transport material layer 05 includes a host material, the host material being a material having hole transport capability;

and the electron transport material layer includes a host material and a phosphorescent dye doped in the host material, the host material being a material having electron transport capability.

In the luminescent layer, the exciplex which is formed between the hole transport material layer 05 and the electron transport material layer 06 meets the following conditions:

T₁^{A}−S₁>0.2 eV

T₁^{D}−S₁≥0.2 eV

|LUMO_{A}|>2.0 eV

|HOMO_{D}|≤5.3 eV

In the above expressions, T₁^{A} represents the first triplet state energy level of the acceptor (the material having electron transport capability), T₁^{D} represents the first triplet state energy level of the donor (the material having hole transport capability), and S₁ represents the first singlet state energy level of the exciplex, LUMO_{A} represents the LUMO energy level of the acceptor, and HOMO_{D} represents the HOMO energy level of the donor.

In another word, the first triplet state energy level of the material having electron transport capability is higher than the singlet state energy level of the exciplex, with an energy gap between the material having electron transport capability and the exciplex being more than or equal to 0.2 eV; and the absolute value of LUMO energy level of the material having hole transport capability is less than or equal to 5.3 eV;

the first triplet state energy level of the material having electron transport capability is higher than the first singlet state energy level of the exciplex, with an energy gap between the material having electron transport capability and the exciplex being more than 0.2 eV; and the absolute value of LUMO energy level of the material having electron transport capability is more than 2.0 eV;

also, the difference in LUMO energy level between the material having hole transport capability and the material having electron transport capability is more than 0.3 eV, preferably more than 0.4 eV;

the difference in HOMO energy level between the material having hole transport capability and the material having electron transport capability is more than 0.2 eV; and

the first singlet state energy level of the exciplex is higher than the first triplet state energy level of the phosphorescent dye.

When the exciplex meets the above conditions, it is a thermally activated delayed delayed fluorescence exciplex (TADF exciplex) and has a thermally activated delayed fluorescence effect. The luminescent layer of the present invention has a double-layer structure in which the TADF exciplex is formed on the interface between the hole transport material layer 05 and the electron transport material layer 06, and the triplet state energy of the TADF exciplex is transferred to the singlet state and then transferred to the dopant dye, so that the triplet state energy of the host material and dopant dye in the device can be fully utilized, thereby increasing the device efficiency; and the energy transfer process and luminescence process of thermally activated delayed fluorescence occur in different materials (named as thermally activated sensitization process), so that the problem of significant roll-off under high luminance conditions can be effectively solved, thereby further improving the stability of the device.

As shown in FIG. 2, in the case of using TCTA as donor and mDTPPC as acceptor, the working principle of the luminescent layer of the present invention will be described below:

The exciplex is generated on the contact interface between the hole transport material layer and the electron transport material layer, so that the excited electrons pass from the first singlet state of the exciplex to the first singlet state of the exciplex by reverse intersystem crossing and then to the first triplet state of the phosphorescent dopant material, emitting a phosphorescent light.

The material having electron transport capability is a compound of the following formulae:

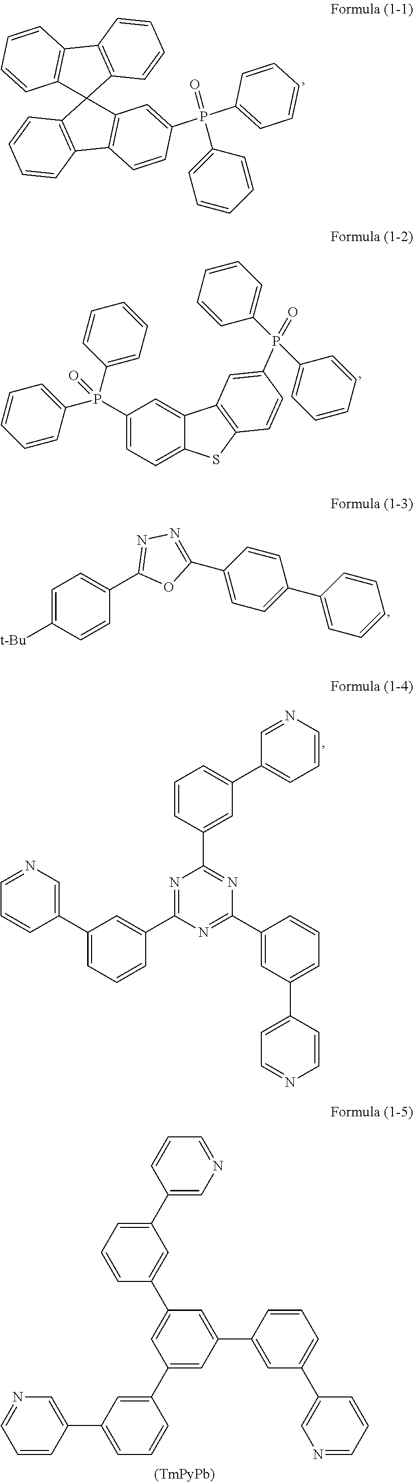 [SMILES: O=P(c1ccccc1)(c1ccccc1)c1ccc2c(c1)C1(c3ccccc3-c3ccccc31)c1ccccc1-2 ; O=P(c1ccccc1)(c1ccccc1)c1ccc2sc3ccc(P(=O)(c4ccccc4)c4ccccc4)cc3c2c1 ; CC(C)(C)c1ccc(-c2nnc(-c3ccc(-c4ccccc4)cc3)o2)cc1 ; c1cncc(-c2cccc(-c3nc(-c4cccc(-c5ccncc5)c4)nc(-c4cccc(-c5cccnc5)c4)n3)c2)c1 ; c1cncc(-c2cccc(-c3cc(-c4cccc(-c5cccnc5)c4)cc(-c4cccc(-c5cccnc5)c4)c3)c2)c1]

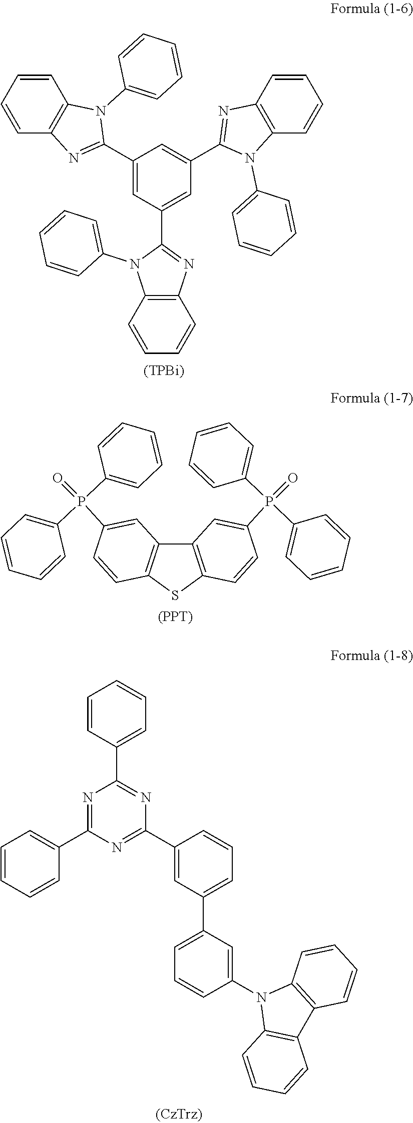

The material having hole transport capability is a compound of the following formulae:

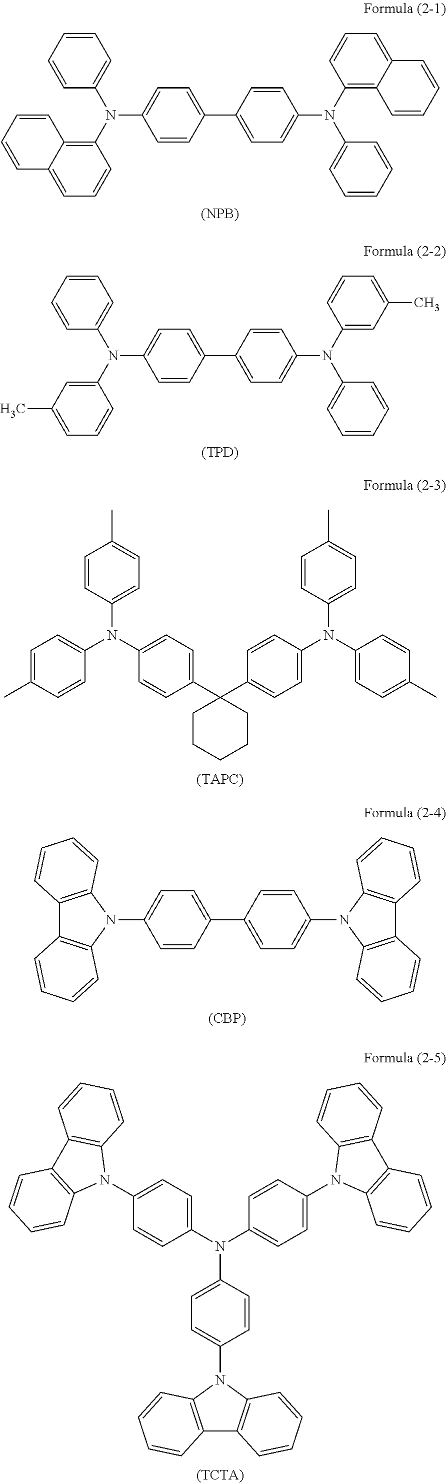

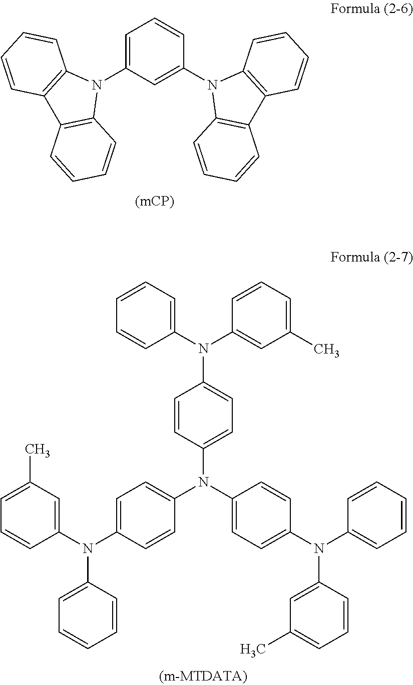

As an example of the organic luminescent display device of the present invention, the anode 01 is made of an inorganic material or organic conductive polymer. The inorganic material is generally a metal oxide such as indium tin oxide (ITO), zinc oxide (ZnO) and indium zinc oxide (IZO), or a metal with higher work functions such as gold, copper and silver, preferably ITO. The organic conductive polymer is preferably one of polythiophene/ polyvinyl sodium benzenesulfonate (hereinafter abbreviated as PEDOT/PSS) and polyaniline (hereinafter abbreviated as PANI).

The cathode 02 is generally made of a metal having lower work functions such as lithium, magnesium, calcium, strontium, aluminum and indium, an alloy thereof with copper, gold or silver, or an electrode layer alternately formed by a metal and a metal fluoride. The cathode 02 in the present invention is preferably a laminate of LiF layer and Al layer (LiF layer on the outer side). The material of the hole transport layer may be selected from aromatic amines and dendrimer-like low molecular materials, preferably NPB.

The material of the electron transport layer may utilize an organic metal complex (such as Alq₃, Gaq₃, BAlq or Ga(Saph-q)) or other materials commonly used in the electron transport layer, such as aromatic fused ring compounds (such as pentacene, germanium) or phenanthrenes (such as Bphen, BCP).

The organic electroluminescence device of the present invention also includes a hole injection layer between the anode 01 and the hole transport layer, and the material of the hole injection layer may be, for example, 4,4′,4″-tri(3-methylphenylaniline)triphenylamine-doped F4TCNQ, or copper phthalocyanine (CuPc), or a metal oxide such as molybdenum oxide and yttrium oxide. The thicknesses of each of the above layers may be conventional for these layers in the art.

The present invention also provides a process for manufacturing the organic electroluminescence device, including successively depositing on the substrate an anode 01, a hole transport layer 03, a luminescent layer 04, an electron transport layer 07 and a cathode 02 in a way of laminating one by one, and then packaging.

The substrate may be a glass or flexible substrate, and the flexible substrate may be made of polyesters or polyimides, or be a thin metal sheet. The laminating and packaging may be completed by any suitable method known to those skilled in the art.

A phosphorescent dye of the luminescent layer in the following examples of the present invention is as follows:

A red phosphorescent dye may be selected from the following compounds:

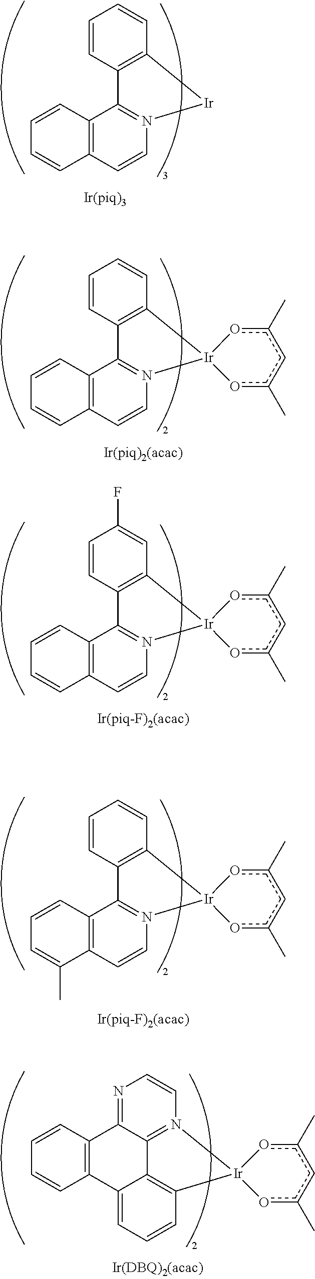

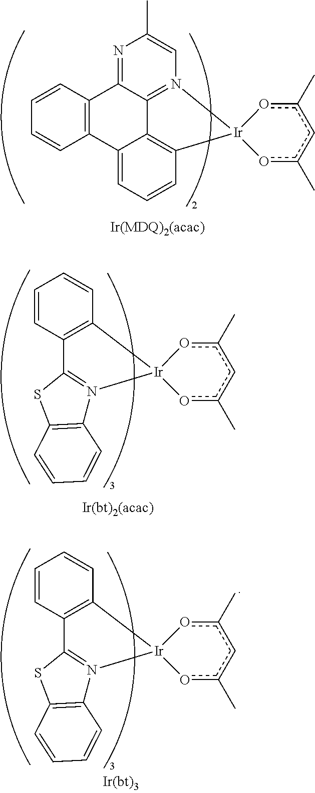

A green phosphorescent dye may be selected from the following compounds:

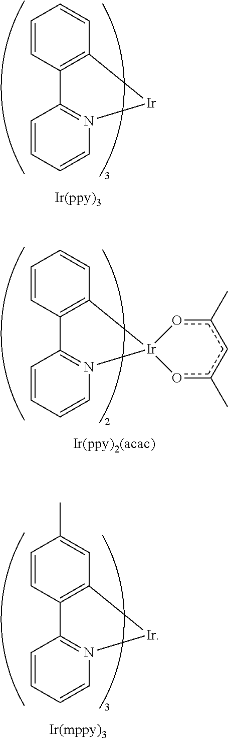

A yellow phosphorescent dye may be selected from the following compounds:

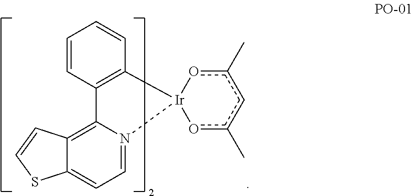

A blue phosphorescent dye used herein may be selected from the following compounds:

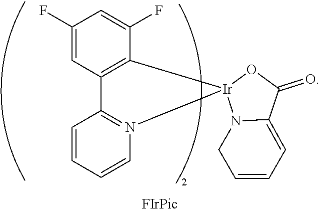

The present invention is further illustrated in connection with the following examples.

### EXAMPLE 1

In this example, luminescent devices in which a hole transport material layer 05 and an electron transport material layer 06 in a luminescent layer have different thicknesses are manufactured, and these devices have a structure shown as FIG. 1. In the luminescent layer, the hole transport material layer 05 consists of a host material compound (2-5) TCTA, and the electron transport material layer 06 consists of a host material compound (1-8) CzTrz and a phosphorescent dye PO-01. The difference in LUMO energy level between the host compound (2-5) TCTA and the acceptor host compound (1-8) CzTrz is more than 0.3 eV, and the difference in HOMO energy is more than 0.2 eV; and the first singlet state energy level of the exciplex is higher than the first triplet state energy level of the phosphorescent dye. The phosphorescent dye PO-01 accounts for 3 wt % of the luminescent layer.

The device of this example has the following structure.

ITO (150 nm)/NPB (40 nm)/hole transport material layer TCTA (10 nm)/electron transport material layer compound (1-8) CzTrz+phosphorescent dye PO-01 (10-40 nm)/Bphen (20 nm)/LiF (0.5nm)/Al (150 nm)

In this example and the description below, the unit of doping concentration of the phosphorescent dye is wt %.

The organic electroluminescence device was manufactured as follows:

first, a glass substrate was cleaned with a detergent and deionized water and placed under an infrared lamp for drying, and a layer of anode material was sputtered on the glass substrate with a film thickness of 150 nm;

then, the glass substrate having the anode 01 was placed in a vacuum chamber under a pressure of 1×10⁻⁴ Pa, and NPB was vapor deposited on the anode film as a hole transport layer 03 under such conditions, the film forming rate was 0.1 nm/s and the vapor deposited film thickness was 40 nm; a luminescent layer was vapor deposited on the hole transport layer 03 by dual-source co-evaporation, in which a hole transport material layer 05TCTA (10 nm) was vapor deposited and then an electron transport material layer 06 CzTrz and a phosphorescent dye PO-01 were vapor deposited at the given mass percentages under a film thickness monitor while the film forming rate was controlled and the vapor deposited film thickness was controlled to be 30 nm;

on the luminescent layer, a layer of Bphen material was further vapor deposited as an electron transport layer 07 under such conditions that the vapor deposition rate was 0.1 nm/s and the total thickness of vapor deposited film was 20 nm; and

finally, a LiF layer and an Al layer were successively vapor deposited as a cathode layer of the device by vapor deposition on the luminescent layer, in which the vapor deposition rate of the LiF layer was 0.01-0.02 nm/s and the thickness was 0.5 nm, and the vapor deposition rate of the Al layer was 1.0 nm/s and the thickness was 150 nm.

### COMPARATIVE EXAMPLE 1

An organic electroluminescence device was manufactured by the same method as Example 1, having the following structure:

ITO (150 nm)/NPB (40 nm)/CBP +3 wt % PO-01(40 nm)/Bphen (20 nm)/LiF (0.5 nm)/Al (150 nm)

In another word, the luminescent layer consists of a host material CBP and a phosphorescent dye PO-01, in which the phosphorescent dye PO-01 accounts for 3 wt % of the luminescent layer. In this example and the description below, the unit of doping concentration of the phosphorescent dye is wt %.

### COMPARATIVE EXAMPLE 2

An organic electroluminescence device was manufactured by the same method as Example 1, having the following structure:

ITO (150 nm)/NPB (40 nm)/exciplex (TCTA and CzTrz, at the mass ratio of 1:1) +3 wt % PO-01 (40 nm)/Alq₃ (20 nm)/LiF (0.5 nm)/Al (150 nm)

In another word, the luminescent layer of this comparative example is a single layer consisting of an exciplex as host material and a phosphorescent dye PO-01 doped in the host material, in which the exciplex consists of a material TCTA having hole transport capability and a material CzTrz having electron transport capability, at the mass ratio of 1:1. The phosphorescent dye PO-01 accounts for 3 wt % of the luminescent layer.

The properties of the organic electroluminescence devices of Example 1 and Comparative example 1 are shown in Table 1 below.

TABLE 1

| | | | | External | |
|---|---|---|---|---|---|
| | | Luminous | | quantum | |
| | Luminescent layer | efficiency | Luminance | efficiency | Lifetime |
| Device | composition | (cd/A) | (cd/m²) | (%) | T₉₇ (hrs) |
| Example 1 | TCTA (10)/CzTrz: 3 wt % | 58 | 1000 | 19 | 462 |
| | PO-01 (10 nm) | | | | |
| | TCTA (10)/CzTrz: 3 wt % | 61 | 1000 | 21 | 471 |
| | PO-01 (20 nm) | | | | |
| | TCTA (10)/CzTrz: 3 wt % | 64 | 1000 | 25 | 480 |
| | PO-01 (30 nm) | | | | |
| | TCTA (10)/CzTrz: 3 wt % | 59 | 1000 | 20 | 465 |
| | PO-01 (40 nm) | | | | |
| Comparative | CBP: 3 wt % PO-01 (40 nm) | 55 | 1000 | 18 | 437 |
| example 1 | | | | | |
| Comparative | TCTA: CzTrz: 3 wt % | 61 | 1000 | 23 | 450 |
| example 2 | PO-01 (40 nm) | | | | |

It can be seen from Table 1 that the luminous efficiencies of the devices of both Example 1 and Comparative example 2, using exciplex as host, are higher than that of Comparative example 1 using the conventional host material CBP, and the lifetime of the device using exciplex as host is longer than that of Comparative example 1. The lifetime of Example 1 is longer than that of Comparative example 2, because in the exciplex system of Comparative example 2, the excited state of the donor or acceptor alone is also formed such that the donor or acceptor is easily decomposed, making the device unstable. In addition, Example 1 adopts a double doping system which is easier for control than a triple doping system adopted by Comparative example 2 in terms of process and is suitable for mass production applications.

### EXAMPLE 2

The device of this example has the following structure.

ITO (150 nm)/NPB (40 nm)/TCTA (10 nm)/CzTrz+1-10 wt % phosphorescent dye PO-01(30 nm)/Bphen (20 nm)/LiF (0.5 nm)/Al (150 nm)

The phosphorescent dye PO-01 accounts for 1-10 wt % of the luminescent layer. In this example, different doping concentrations of phosphorescent dye were applied in the luminescent layer for experiments, and the results were shown in Table 2.

TABLE 2

| | | | External | |
|---|---|---|---|---|
| | Luminous | | quantum | Lifetime |
| | efficiency | Luminance | efficiency | T97 |
| Luminescent layer | (cd/A) | (cd/m²) | (%) | (hrs) |
| TCTA (10)/CzTrz: | 58 | 1000 | 14.1 | 435 |
| 1 wt % PO-01 (30 nm) | | | | |
| TCTA (10)/CzTrz: | 61 | 1000 | 14.7 | 462 |
| 2 wt % PO-01 (30 nm) | | | | |
| TCTA (10)/CzTrz: | 64 | 1000 | 16.1 | 480 |
| 3 wt % PO-01 (30 nm) | | | | |
| TCTA (10)/CzTrz: | 57 | 1000 | 14.6 | 471 |
| 5 wt % PO-01 (30 nm) | | | | |
| TCTA (10)/CzTrz: | 54 | 1000 | 12.9 | 443 |
| 10 wt % | | | | |
| PO-01 (30 nm) | | | | |

It can be seen from Table 2 that when the doping concentration of the phosphorescent dye in the luminescent layer is 3 wt %, both the external quantum efficiency and the lifetime of the OLED device are optimal.

### EXAMPLE 3

In order to evaluate the influence of the host material on the performance of the organic electroluminescence device of the present invention, an organic electroluminescence device was manufactured by the same method as Example 1, having the following structure:

ITO (150 nm)/NPB (40 nm)/hole transport material layer (material having hole transport capability) (10 nm)/electron transport material layer (material having electron transport capability +3 wt % phosphorescent dye) (30 nm)/Bphen (20nm)/LiF (0.5nm)/Al (150 nm)

The performance of the organic electroluminescence device is shown in Table 3 below:

TABLE 3

| | | | Luminous | | External quantum | Lifetime |
|---|---|---|---|---|---|---|
| | Hole transport | Electron transport | efficiency | Luminance | efficiency | T97 |
| Device | material layer | material layer | (cd/A) | (cd/m²) | (%) | (hrs) |
| OLED1 | compound | compound (1-3) + | 23 | 1000 | 18 | 390 |
| | (2-7) | phosphorescent | | | | |
| | | dye Ir(piq)₃ | | | | |
| OLED2 | compound | compound (1-7) + | 20 | 1000 | 16 | 348 |
| | (2-7) | phosphorescent | | | | |
| | | dye Ir(DBQ)₂(acac) | | | | |
| OLED3. | compound | compound (1-1) + | 67 | 1000 | 17 | 390 |
| | (2-5) | phosphorescent | | | | |
| | | dye Ir(ppy)₃ | | | | |
| OLED4 | compound | compound (1-3) + | 63 | 1000 | 16 | 420 |
| | (2-1) | phosphorescent | | | | |
| | | dye Ir(mppy)₃ | | | | |
| OLED5. | compound | compound (1-5) + | 38 | 1000 | 14 | 65 |
| | (2-2) | phosphorescent | | | | |
| | | dye FIrPic | | | | |

It can be seen from Table 3 that the devices of OLED1 to OLED5 in which the exciplex is formed as host on the interface between the hole transport material layer 05 and the electron transport material layer 06 have excellent electrical properties and increased lifetime, indicating that the triplet state energy of the TADF exciplex that is formed on the interface between the hole transport material layer 05 and the electron transport material layer 06 is transferred to the singlet state and then transferred to the triplet state of the dopant material, so that the triplet state energy of both the host material and the dopant material are fully utilized, thereby improving the device efficiency. The above embodiments are merely preferred embodiments for fully describing the present invention, and not intended to limit the scope of the present invention. Any equivalents or changes made by those skilled in the art on the basis of the present invention fall within the scope of the present invention. The scope of the present invention is defined by the claims.

1. A phosphorescent organic electroluminescence device, including a hole transport layer, a luminescent layer and an electron transport layer, which are successively laminated, wherein the luminescent layer has a double-layer structure comprising a hole transport material layer and an electron transport material layer, the hole transport material layer is arranged between the hole transport layer and the electron transport material layer, the electron transport material layer is arranged between the hole transport material layer and the electron transport layer, and an exciplex is formed on a contact interface between the hole transport material layer and the electron transport material layer;
the hole transport material layer includes a host material having hole transport capability; and
the electron transport material layer includes a host material and a phosphorescent dye doped in the host material, the host material 1 having electron transport capability;
wherein, the first triplet state energy level of the material having electron transport capability is higher than the singlet state energy level of the exciplex, with an energy gap between the material having electron transport capability and the exciplex being more than or equal to 0.2 eV;
and the absolute value of HOMO energy level of the material having hole transport capability is less than or equal to 5.3 eV;
the first triplet state energy level of the material having electron transport capability is higher than the first singlet state energy level of the exciplex, with an energy gap between the material having electron transport capability and the exciplex being more than 0.2 eV; and the absolute value of LUMO energy level of the material having electron transport capability is more than 2.0 eV; the difference in LUMO energy level between the material having hole transport capability and the material having electron transport capability is more than 0.3 eV, and the difference in HOMO energy level between the material having hole transport capability and the material having electron transport capability is more than 0.2 eV; and
the first singlet state energy level of the exciplex is higher than the first triplet state energy level of the phosphorescent dye.

2. The phosphorescent organic electroluminescence device according to claim 1, wherein the difference in LUMO energy level between the material having hole transport capability and the material having electron transport capability is more than or equal to 0.4 eV.

3. The phosphorescent organic electroluminescence device according to claim 1, wherein the phosphorescent dye accounts for 1-10 wt % of the luminescent layer.

4. The phosphorescent organic electroluminescence device according to claim 1, wherein the phosphorescent dye accounts for 3 wt % of the luminescent layer.

5. The phosphorescent organic electroluminescence device according to claim 1, wherein the thickness ratio of the hole transport material layer to the electron transport material layer is 1:1 to 1:5.

6. The phosphorescent organic electroluminescence device according to claim 1, wherein the thickness ratio of the hole transport material layer to the electron transport material layer is 1:3.

7. The phosphorescent organic electroluminescence device according to claim 1, wherein the material having electron transport capability is one or more of compounds of the structural formulae (1-1) to (1-8):

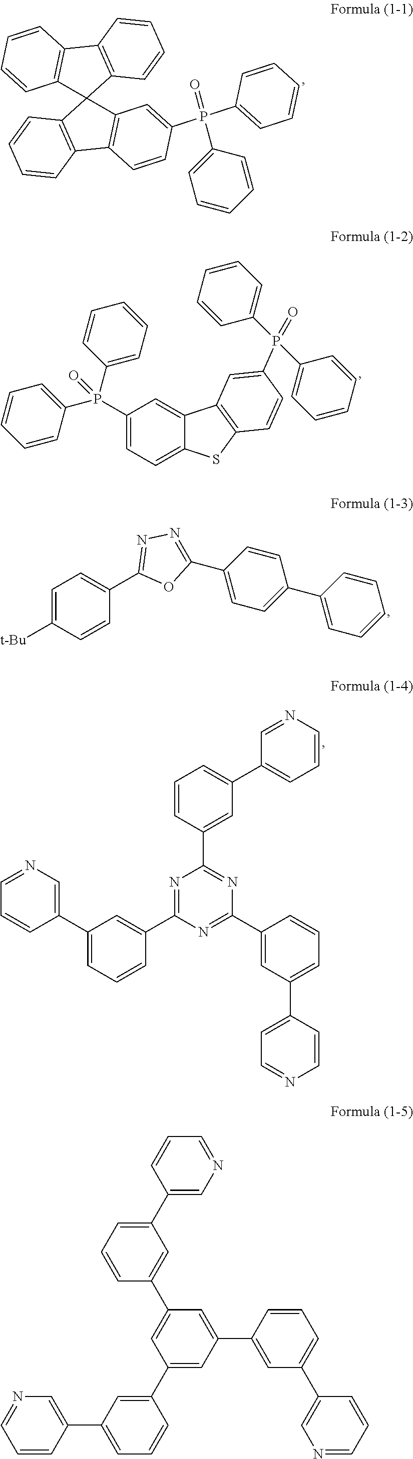 [SMILES: O=P(c1ccccc1)(c1ccccc1)c1ccc2c(c1)C1(c3ccccc3-c3ccccc31)c1ccccc1-2 ; O=P(c1ccccc1)(c1ccccc1)c1ccc2sc3ccc(P(=O)(c4ccccc4)c4ccccc4)cc3c2c1 ; CC(C)(C)c1ccc(-c2nnc(-c3ccc(-c4ccccc4)cc3)o2)cc1 ; c1cncc(-c2cccc(-c3nc(-c4cccc(-c5ccncc5)c4)nc(-c4cccc(-c5cccnc5)c4)n3)c2)c1 ; c1cncc(-c2cccc(-c3cc(-c4cccc(-c5cccnc5)c4)cc(-c4cccc(-c5cccnc5)c4)c3)c2)c1]

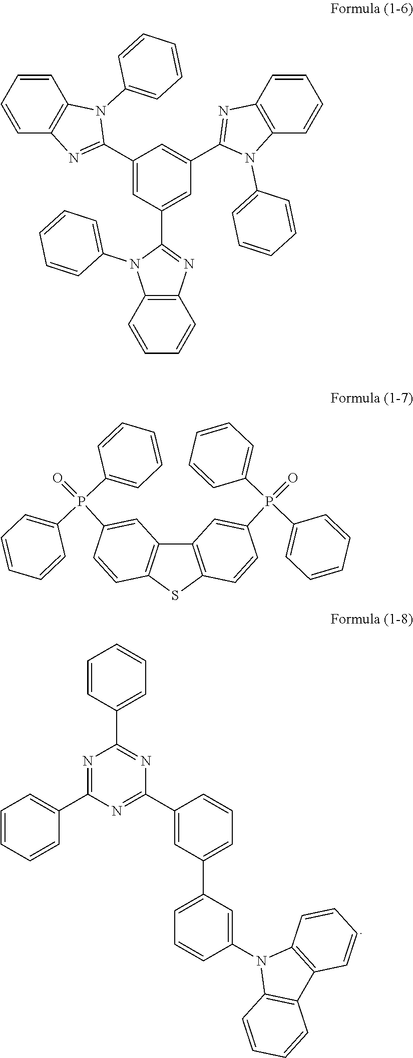 [SMILES: c1ccc(-n2c(-c3cc(-c4nc5ccccc5n4-c4ccccc4)cc(-c4nc5ccccc5n4-c4ccccc4)c3)nc3ccccc32)cc1 ; O=P(c1ccccc1)(c1ccccc1)c1ccc2sc3ccc(P(=O)(c4ccccc4)c4ccccc4)cc3c2c1 ; c1ccc(-c2nc(-c3ccccc3)nc(-c3cccc(-c4cccc(-n5c6ccccc6c6ccccc65)c4)c3)n2)cc1]

8. The phosphorescent organic electroluminescence device according to claim 1, wherein, the material having hole transport capability is one or more of compounds of the structural formulae (2-1) to (2-7):

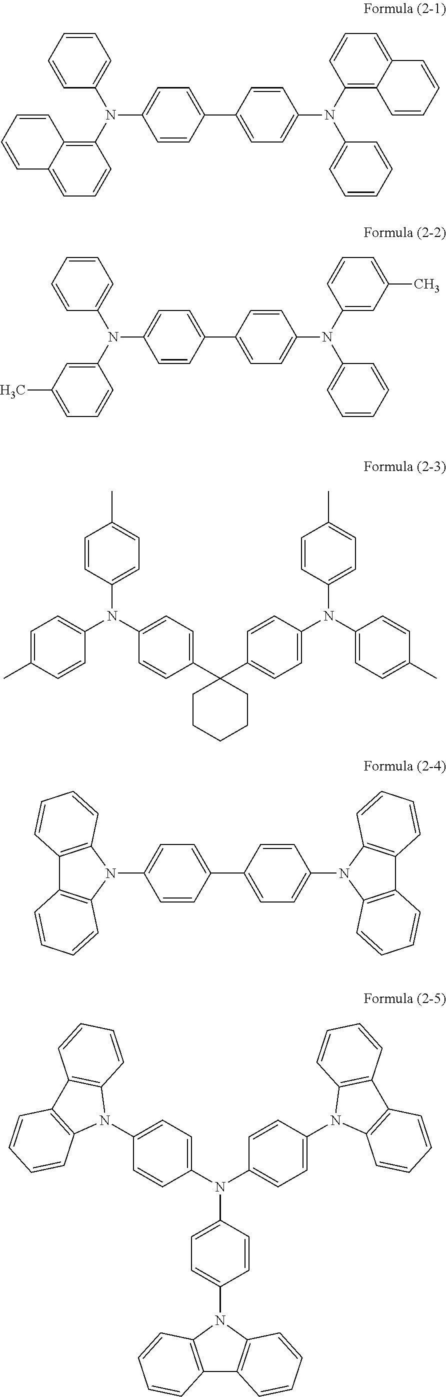

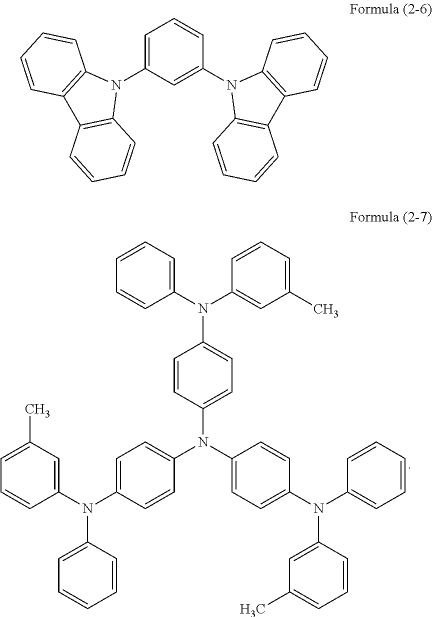

9. The phosphorescent organic electroluminescence device according to claim 1, wherein the phosphorescent organic electroluminescence device includes an anode, a hole transport layer, the hole transport material layer, the electron transport material layer, an electron transport layer and a cathode, which are successively laminated on a substrate.

10. The phosphorescent organic electroluminescence device according to claim 9, wherein the anode and the hole transport layer has a hole injection layer arranged therebetween.
